(19) United States
(12) Patent Application Publication
(10) Pub. No.: US 2022/0287733 A1
(43) Pub. Date: Sep. 15, 2022

(54) DEVICES, SYSTEMS, AND METHODS FOR DISRUPTING OBSTRUCTIONS IN BODY LUMENS

(71) Applicant: Covidien LP, Mansfield, MA (US)

(72) Inventor: James Davidson, San Juan Capistrano, CA (US)

(21) Appl. No.: 17/249,830

(22) Filed: Mar. 15, 2021

(51) Int. Cl. A61B 17/225; A61M 5/00

(52) U.S. Cl. CPC A61B 17/2256; A61M 5/007; A61B 2090/376

(57) ABSTRACT

Systems and methods for detecting and disrupting obstructions (such as clot material) within a blood vessel are disclosed herein. In some examples, the present technology comprises a system for detecting and disrupting a clot in a cerebral blood vessel of a patient, where the system comprises a treatment environment, a detection system, and an energy delivery device. The detection system may be configured to determine the presence of a blood clot within a cerebral blood vessel of a patient. In some embodiments, the detection system is configured to obtain data characterizing a position of the clot within the treatment environment. The energy delivery device can be configured to receive the data characterizing the position of the clot and, based on the data, deliver energy to the clot, thereby disrupting the clot and restoring blood flow in the affected blood vessel.

### TECHNICAL FIELD

The present technology relates to devices, systems, and methods for disrupting obstructions in body lumens. In particular, the present technology is directed to devices, systems, and methods for detecting and disrupting clot material in blood vessels.

### BACKGROUND

Stroke is a serious medical condition that can cause permanent neurological damage, complications, and death. Ischemic stroke is the result of a thrombus or embolus obstructing blood flow in a cerebral blood vessel and thus to brain tissue, leading to dysfunction of the affected brain tissue. As approximately two million neurons die each minute following onset of a stroke, it is critical that the stroke be diagnosed and treated as quickly as possible to preserve function of the affected brain tissue.

A variety of approaches exist for treating patients experiencing an ischemic stroke. For example, a clinician may administer anticoagulants (e.g., warfarin) or thrombolytic agents (e.g., tissue plasminogen activator (tPA)), or may undertake intravascular interventions such as mechanical thrombectomy procedures. However, such approaches suffer from certain drawbacks. For example, there is a limited window in which thrombolytic agents can be administered following stroke onset. Further, thrombolytic agents such as tPA have limited efficacy in treating large vessel occlusions and may cause adverse events if improperly administered to a patient experiencing a hemorrhagic stroke. While mechanical thrombectomy procedures have a longer administration window and can be more effective than thrombolytic agents alone in some cases, such procedures require advanced imaging, specific equipment, and highly skilled and trained clinicians. Consequently, mechanical thrombectomy is not an accessible treatment option for many patients. Further, delays in time to treatment by mechanical thrombectomy as a result of time required to transport a patient to an appropriate facility and prepare the patient for the procedure can result in greater neurological damage, disability, and mortality. Accordingly, improved systems and methods for treating stroke are needed.

### SUMMARY

The present technology relates to systems and methods for disrupting obstructions such as clot material in body lumens. In particular embodiments, the present technology relates to systems and methods for noninvasively detecting and disrupting blood clots in cerebral blood vessels. The subject technology is illustrated, for example, according to various aspects described below, including with reference to FIGS. 1-6. Various examples of aspects of the subject technology are described as numbered clauses (1, 2, 3, etc.) for convenience. These are provided as examples and do not limit the subject technology.

1. A method comprising:
positioning a reference marker proximate a head of a patient, the patient having a clot within a cerebral blood vessel;
obtaining first data characterizing a position of the reference marker relative to a coordinate system;
obtaining second data characterizing a position of the clot relative to the position of the reference marker;
based on the first data and the second data, determining a position of the clot relative to the coordinate system; and
based on the position of the clot relative to the coordinate system, delivering focused energy from an extracorporeally-positioned energy delivery device to the clot, thereby disrupting the clot.

2. The method of Clause 1, wherein the first data comprises three-dimensional coordinates.

3. The method of Clause 1 or Clause 2, wherein the second data comprises a three-dimensional position vector.

4. The method of any one of Clauses 1 to 3, wherein the reference marker is a first reference marker, the method further comprising positioning second and third reference markers proximate a head of the patient.

5. The method of any one of Clauses 1 to 4, wherein the reference marker is extracorporeally-positioned.

6. The method of any one of Clauses 1 to 5, wherein the reference marker is retroreflective.

7. The method of any one of Clauses 1 to 6, wherein the reference marker is radiopaque.

8. The method of any one of Clauses 1 to 7, wherein obtaining the first data comprises obtaining an image of the reference marker using an optical camera system.

9. The method of Clause 8, wherein the optical camera system comprises at least two cameras.

10. The method of any one of Clauses 1 to 9, wherein obtaining the first data comprises determining three-dimensional coordinates of the reference marker using a position sensing device.

11. The method of any one of Clauses 1 to 10, wherein obtaining the first data comprises obtaining an image of the reference marker using a medical imaging device.

12. The method of Clause 11, wherein the medical imaging device uses a modality comprising x-ray, fluoroscopy, magnetic resonance imaging, computed tomography, ultrasound, positron emission tomography, single photon emission coherence tomography, optical coherence tomography, magnetic particle imaging, or magnetic particle spectroscopy.

13. The method of Clause 11 or Clause 12, wherein obtaining the second data comprises obtaining an image of the patient and the clot using the medical imaging device.

14. The method of any one of Clauses 1 to 13, further comprising marking the clot with a marking agent.

15. The method of Clause 14, wherein the marking agent is a biomarker.

16. The method of Clause 15, wherein the biomarker is a peptide.

17. The method of Clause 16, wherein the peptide is a fibrin-binding peptide.

18. The method of Clause 14, wherein the marking agent is a nanoparticle.

19. The method of Clause 18, wherein the nanoparticle is an iron oxide nanoparticle.

20. The method of Clause 18, wherein the nanoparticle is a gold nanoparticle.

21. The method of Clause 14, wherein the marking agent is a contrast agent.

22. The method of Clause 21, wherein the contrast agent comprises microbubbles or a radiotracer.

23. The method of any one of Clauses 14 to 22, wherein marking the clot comprises intravenously administering the marking agent to the patient.

24. The method of any one of Clauses 1 to 23, wherein positioning the reference marker, obtaining first data, obtaining second data, determining a position of the clot relative to the coordinate system, delivering energy, and marking the clot occur within a vehicle.

25. The method of Clause 24, wherein the vehicle is an ambulance.

26. The method of any one of Clauses 1 to 25, further comprising administering a fibrinolytic agent to the patient.

27. The method of any one of Clauses 1 to 26, further comprising intravenously administering a cavitation-facilitating agent to the patient.

28. The method of any one of Clauses 1 to 27, wherein the energy delivery device is a high-intensity focused ultrasound device.

29. The method of any one of Clauses 1 to 28, further comprising modifying a position or an orientation of the patient based on the position of the clot relative to the coordinate system.

30. The method of any one of Clauses 1 to 29, further comprising modifying a position or an orientation of the energy delivery device based on the position of the clot relative to the coordinate system.

31. The method of any one of Clauses 1 to 30, further comprising modifying a parameter of the energy delivery device based on the position of the clot relative to the coordinate system.

32. The method of Clause 31, wherein the parameter comprises a frequency, an acoustic power, a pulse width, a pulse duration, a number of pulses, or a treatment duration.

33. A method comprising:
positioning a patient within a treatment environment, the patient having a clot within a blood vessel;
marking the clot with a marking agent;
determining a relationship between a local coordinate system of a detection system and a global coordinate system of the treatment environment;
obtaining data characterizing a position of the clot relative to the global coordinate system of the treatment environment with the detection system;
based on the data, delivering focused energy from an extracorporeally-positioned energy delivery device to the clot, thereby disrupting the clot.

34. The method of Clause 33, wherein obtaining the data characterizing the position of the clot relative to the global coordinate system comprises obtaining data characterizing the position of the clot relative to the local coordinate system of the detection system and, based on the relationship between the local and global coordinate systems, determining the position of the clot relative to the global coordinate system.

35. The method of Clause 33 or Clause 34, wherein the relationship comprises a transformation matrix.

36. The method of any one of Clauses 33 to 35, wherein the data comprises three-dimensional coordinates.

37. The method of any one of Clauses 33 to 36, wherein the detection system comprises a medical imaging device.

38. The method of any one of Clauses 33 to 37, wherein the marking agent comprises a peptide, a nanoparticle, or a contrast agent.

39. The method of any one of Clauses 33 to 38, wherein the focused energy is high-intensity focused ultrasound energy.

40. The method of any one of Clauses 33 to 39, further comprising positioning a reference marker proximate the patient.

41. The method of Clause 40, further comprising obtaining data characterizing a position of the reference marker relative to the global coordinate system of the treatment environment with the detection system.

42. A non-transitory computer readable medium having stored thereon instructions executable by a computing device to cause the computing device to perform functions comprising:
obtaining first data characterizing a position of a reference marker relative to a coordinate system;
obtaining second data characterizing a position of a thrombus within a blood vessel of a patient relative to the position of the reference marker;
based on the first data and the second data, determining a position of the thrombus relative to the coordinate system; and
based on the position of the thrombus relative to the coordinate system, causing an extracorporeally-positioned energy delivery device to deliver focused energy to the thrombus to thereby fragment the clot.

43. The non-transitory computer-readable medium of Clause 42, wherein the first data comprises three-dimensional coordinates.

44. The non-transitory computer-readable medium of Clause 42 or Clause 43, wherein the second data comprises a three-dimensional position vector.

45. The non-transitory computer-readable medium of Clause 42 or Clause 43, wherein the second data comprises three-dimensional coordinates.

46. The non-transitory computer-readable medium of any one of Clauses 42 to 45, wherein causing the energy delivery device to deliver focused energy to the treatment location comprises modifying a position of the energy delivery device.

47. The non-transitory computer-readable medium of any one of Clauses 42 to 46, wherein causing the energy delivery device to deliver focused energy to the treatment location comprises modifying a parameter of the energy delivery device.

48. A method for treating a patient in a treatment environment, the patient having a blood clot, wherein the method comprises:
marking the clot with a marking agent;
positioning a reference marker on the patient;
imaging the marked clot and the reference marker to obtain location data corresponding to a location of the clot within the treatment environment; and
delivering focused energy from an extracorporeally-positioned energy delivery device to the location, thereby disrupting the clot.

49. The method of Clause 48, wherein the reference marker is a first reference marker and the method further comprises:
positioning a second reference marker on the patient;
imaging the marked clot and the first and second reference markers to obtain position information characterizing the positions of the marked clot, the first reference marker, and the second reference marker relative to one another;
triangulating the location of the clot using the position information.

50. The method of Clause 48 or Clause 49, further comprising determining whether the patient is suffering a hemorrhagic or ischemic stroke based on the imaging.

51. The method of any one of Clauses 48 to 50, further comprising delivering itPA to the clot prior to delivering the focused energy.

52. The method of any one of Clauses 48 to 51, wherein the marking agent is a thrombus-binding peptide or nano-particles.

53. The method of any one of Clauses 48 to 52, wherein the focused energy is high-intensity focused ultrasound (HIFU).

54. The method of any one of Clauses 48 to 53, wherein the time to perform the method is about 1 to 2 hours.

55. The method of any one of Clauses 48 to 54, wherein the clot is a cerebral blood clot and the reference marker is positioned on the patient's head.

56. The method of any one of Clauses 48 to 55, wherein the marked clot and the reference marker are imaged with a CT scanner or an ultrasound scanner.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic diagram of a system for disrupting an obstruction in a blood vessel of a patient configured in accordance with several embodiments of the present technology.

FIG. 2 is a schematic diagram of a detection system configured in accordance with several embodiments of the present technology.

FIG. 3 depicts an energy delivery device configured in accordance with several embodiments of the present technology.

FIG. 4 is a schematic diagram of a detection system configured in accordance with several embodiments of the present technology.

FIG. 5 is a schematic diagram of a detection system configured in accordance with several embodiments of the present technology.

FIG. 6 is a schematic diagram of a process flow for disrupting an obstruction in a blood vessel of a patient in accordance with several embodiments of the present technology.

### DETAILED DESCRIPTION

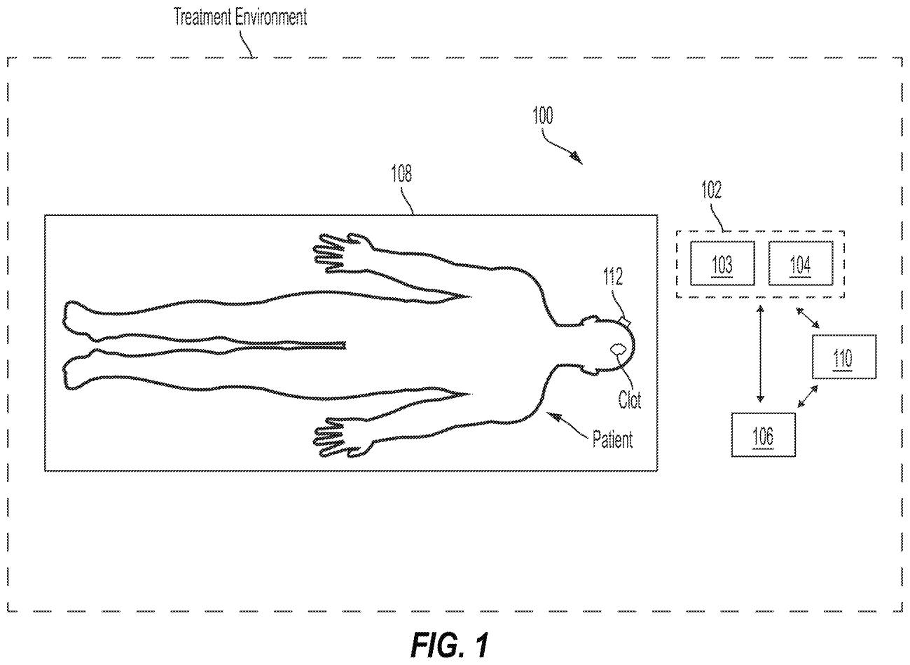

FIG. 1 depicts a system 100 within a treatment environment according to several embodiments of the present technology. The system 100 may be configured to locate and disrupt an obstruction (such as clot material) within a body lumen of a patient. As used herein, “disruption” includes any reduction in size, break up, disintegration, and/or removal of the obstruction via one or more of lysis, fragmentation, maceration, dissolution, digestion, and others. In some embodiments, for example as shown in FIG. 1, the system 100 comprises a detection system 102 and an energy delivery device 106. The detection system 102 can be configured to locate an obstruction, such as clot material, within a blood vessel of a patient. For example, the detection system 102 may be configured to obtain data characterizing a position of the clot material within the treatment environment. In some embodiments, the data comprises the three-dimensional coordinates of the clot material within the treatment environment. The energy delivery device 106 can be configured to receive the data characterizing the position of the clot material and, based on the data, deliver energy to the clot material, thereby disrupting the clot material and improving and/or restoring blood flow in the affected blood vessel.

The system 100 of the present technology is shown in FIG. 1 in use within a treatment environment. Current approaches for treating ischemic stroke (e.g., administration of thrombolytic agents, mechanical thrombectomy, etc.) must be performed at a hospital, and the delay in treatment resulting from the travel time to the hospital may cause greater neurological damage or other adverse events. To reduce and/or eliminate such delays, the system 100 of the present technology enables stroke treatment in a mobile setting, such as an ambulance or helicopter. As a result, the devices and systems of the present technology can treat a patient suffering from a stroke more rapid1y. The treatment environment, for example, may comprise an ambulance, helicopter, boat or other vehicle containing the detection system 102 and the energy delivery device 106 of the present technology.

The detection system 102 and/or energy delivery device 106 may have a fixed position within the treatment environment, or may be movable within the treatment environment. In some embodiments, the detection system 102 and the energy delivery device 106 are integrated into a single device, and in some embodiments the detection system 102 and energy delivery device 106 are distinct components that are movable relative to one another.

In some embodiments, for example as shown in FIG. 1, the treatment environment includes a positioning device 108 configured to maintain the patient at a fixed location and/or orientation relative to the treatment environment. The positioning device 108 can comprise any suitable structure including, but not limited to, a bed, a table, or a chair. In some embodiments, for example when facilitating treatment of a patient with a thrombus in a cerebral blood vessel, the positioning device 108 comprises a head immobilizer configured to maintain a head of the patient at a fixed location and/or orientation relative to the treatment environment. The head immobilizer can comprise one or more straps, a mask, a support, a block, a wedge, a frame, an air bladder, a crad1e, or another suitable immobilization or positioning structure.

The detection system 102 may be configured to determine the position of clot material within the treatment environment, such as the three-dimensional coordinates of the clot material. In some embodiments, the detection system 102 comprises a first data collector 103 configured to obtain data characterizing the position of the clot (“clot position data”). The first data collector 103 can include any suitable device or collection of devices configured to obtain the clot position data. In some embodiments, the first data collector 103 is a medical imaging device comprising a modality such as, but not limited to, computed tomography (CT), magnetic resonance imaging (MRI), ultrasound, x-ray, fluoroscopy, angiography, positron emission tomography (PET), single photon emission coherence tomography (SPECT), optical coherence tomography (OCT), or magnetic particle imaging (MPI). Additionally or alternatively, the first data collector 103 may include a non-imaging measurement modality including, but not limited to, metal detection, electromagnetic sensing, inductive proximity sensing, capacitive proximity sensing, eddy current sensing, hall effect sensing, or magnetic particle spectroscopy.

According to some embodiments, the system 100 includes a marking agent configured to be delivered to the affected blood vessel proximate the clot material to facilitate identification and/or visualization of the clot material by the first data collector 103. The marking agent can be a nanoparticle (e.g., a gold nanoparticle, an iron oxide nanoparticle, etc.), a biomarker (e.g., a fibrin-binding peptide, etc.), a contrast agent (e.g., microbubbles, radiotracers, etc.), or others. In some embodiments, properties of the marking agent are based, at least in part, on the first data collector 103 modality. For example, the marking agent can be a gold nanoparticle when the first data collector 103 modality is CT, an iron oxide nanoparticle when the first data collector 103 modality is MPI, a radiotracer when the first data collector 103 modality is PET, etc. According to some embodiments, the marking agent is configured to be administered intravenously to the patient.

In some embodiments, the detection system 102 comprises a second data collector 104 configured to obtain data characterizing the position of a reference marker 112 within the treatment environment (“reference position data”). The second data collector 104 can include any suitable device or collection of devices configured to obtain the reference position data. The second data collector 104 may comprise a modality such as, but not limited to, optical imaging, optical proximity sensing, time of flight sensing, medical imaging, or a non-imaging measurement modality as described elsewhere herein. Properties of the reference marker 112 may be based, at least in part, on the second data collector 104 modality. For example, the reference marker 112 may be retroreflective for use with an optical imaging system or radiopaque for use with an a radiographic (e.g., x-ray, CT) imaging system.

In some embodiments, the reference marker 112 is positioned extracorporeally. For example, the reference marker 112 can be positioned proximate the head of the patient. In some embodiments, for example as shown in FIG. 1, the reference marker 112 may be removably adhered or coupled to a patient's head such that the position and/or orientation of the reference marker 112 is fixed relative to the patient's head. Additionally or alternatively, the position of the reference marker 112 may be fixed within the treatment environment. The detection system 102 may comprise a single reference marker 112, two reference markers 112, three reference markers 112, or more. In some embodiments, the detection system 102 does not comprise a reference marker 112.

According to some embodiments, the system 100 further comprises a computing device 110. The computing device 110 can be communicatively coupled to the detection system 102 and/or the energy delivery device 106. For example, the computing device 110 can be communicatively coupled to the first data collector 103 and/or the second data collector 104. Additionally or alternatively, the first data collector 103, the second data collector 104, and/or the energy delivery device 106 can each comprise a collection of devices in which one or more of the devices is a computing device. A computing device in accordance with the present technology can be any suitable combination of software and hardware. For example, the computing device can include a special purpose computer or data processor that is specifically programmed, configured, or constructed to perform one or more of the computer-executable instructions explained in detail herein. Additionally or alternatively, the computing device can include a distributed computing environment in which tasks or modules are performed by remote processing devices, which are linked through a communication network (e.g., a wireless communication network, a wired communication network, a cellular communication network, the Internet, a short-range radio network (e.g., via Bluetooth)). In a distributed computing environment, program modules may be located in both local and remote memory storage devices.

Computer-implemented instructions, data structures, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In some embodiments, aspects of the technology may be distributed over the Internet or over other networks (e.g. a Bluetooth network) on a propagated signal on a propagation medium (e.g., an electromagnetic wave(s), a sound wave) over a period of time, or may be provided on any analog or digital network (packet switched, circuit switched, or other scheme).

The system 100 can also include one or more input devices (e.g., touch screen, keyboard, mouse, microphone, camera, etc.) and/or one or more output devices (e.g., display, speaker, etc.) coupled to the computing device. In operation, a user can provide instructions to the computing device and receive output from the computing device via the input and output devices.

The energy delivery device 106 of the present technology can be configured to deliver energy to the clot and thereby disrupt the clot and restore blood flow in the previously obstructed blood vessel. In some embodiments, the energy delivery device 106 is a high-intensity focused ultrasound (HIFU) device configured to deliver focused ultrasound energy to the clot material. The energy delivery device 106 may be positioned extracorporeally. The energy delivery device 106 can be configured to receive data characterizing the position of the clot in the treatment environment from the detection system 102 and/or the computing device 110. Based on the data, a position, an orientation, and/or a parameter of the energy delivery device 106 can be modified to direct the energy to the clot material.

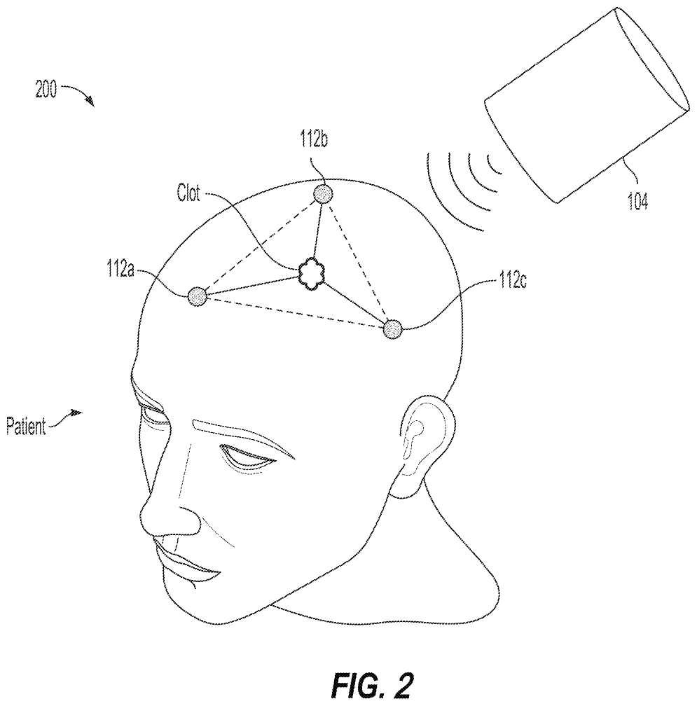

FIG. 2 depicts a detection system 200 in accordance with some embodiments of the present technology. The system 100 may comprise one or more reference markers (labeled individually as 112a, 112b, and 112c), an imaging device (not shown), and an energy delivery device 104, such as a HIFU device. A method for using the system 100 to treat a patient having a clot, such as a cerebral clot, can comprise, for example, marking the clot with a marking agent. The marking agent can be a thrombus-binding peptide or nano-particle, or other suitable agent. The method can further include positioning a reference marker (such as reference marker 112a) on the patient and imaging the marked clot and the reference marker to obtain location data corresponding to a location of the clot within the treatment environment. The reference marker can be placed on the patient's head and/or another location on the patient's body. The imaging can be performed with a CT scanner, an ultrasound scanner, or other imaging device. In some embodiments, the method optionally includes determining whether the patient is suffering a hemorrhagic or ischemic stroke based on the imaging. According to several embodiments, the method comprises delivering itPA to the clot prior to delivering the focused energy.

The method can continue with delivering focused energy from the extracorporeally-positioned energy delivery device 104 to the location, thereby disrupting the clot. In some embodiments, the reference marker is a first reference marker 112a and the method further comprises positioning a second reference marker 112b on the patient. In such embodiments, the method may proceed with imaging the marked clot and the first and second reference markers 112a, 112b to obtain position information characterizing the positions of the marked clot, the first reference marker 112a, and the second reference marker 112b relative to one another. The method can further include triangulating the location of the clot using the position information.

In any of the embodiments disclosed herein, the time to perform the method is about 1 to 2 hours, which is significantly faster than the current standard of care for stroke patients.

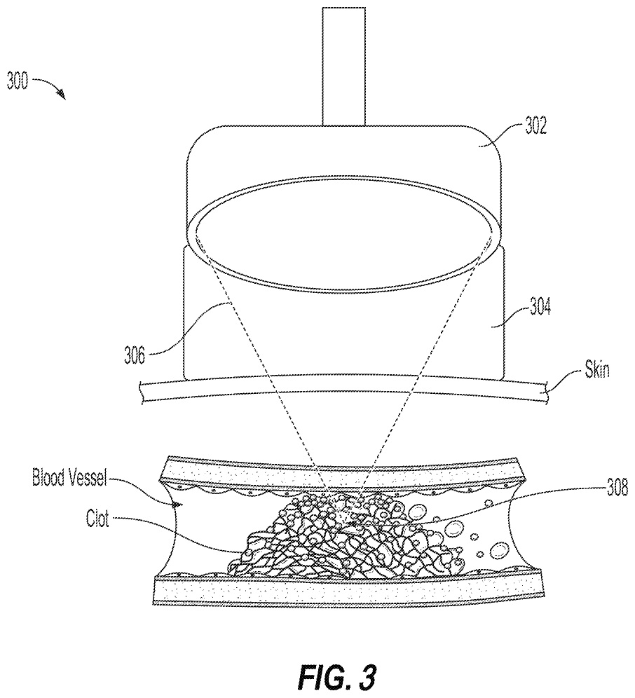

FIG. 3 depicts an energy delivery device 300 configured in accordance with several embodiments of the present technology. The energy delivery device 300 can be used with any of the embodiments of the system disclosed herein, or with other systems. In some embodiments, the energy delivery device 300 is configured to deliver HIFU energy to an obstruction, such as clot material, in a blood vessel of a patient in order to disrupt the obstruction. As shown in FIG. 3, the energy delivery device 300 can comprise a transducer 302 positioned extracorporeally and proximate the patient. The transducer 302 may comprise a spherically curved transducer, a flat transducer with an interchangeable lens, a phased-array transducer, or any other suitable transducer configured to generate focused ultrasound beams. A coupling agent 304 (e.g., water) can be positioned between the transducer 302 and the patient to facilitate transmission of the energy from the transducer 302 into the patient to reach the clot material. In some embodiments, the coupling agent is degas sed to minimize the formation of cavitation bubbles in the coupling agent, which may interfere with the energy transmission. The transducer 302 can be coupled to an ultrasound driving system configured to generate an ultrasound beam 306 based on specified input parameters. The ultrasound driving system may include a computing device, as described elsewhere herein. The ultrasound beam 306 can be configured to pass through skin of the patient and converge at a focal point 308. The energy delivery device 300 can be configured such that a position of the focal point 308 relative to a coordinate system of the treatment environment is the same as a position of the clot material relative to the coordinate system of the treatment environment. Based on the position of the clot material within the treatment environment, the position, orientation, and/or one or more parameters of the energy delivery device 300 can be modified to align the focal point 308 of the ultrasound beam 306 with the clot material. The parameters include, but are not limited to, frequency, acoustic power, number of pulses, pulse duration, and treatment duration. In some embodiments, the position and/or orientation of the patient are modified to align the focal point 308 with the clot material, rather than the position and/or orientation of the energy delivery device 300. A cavitation-facilitating agent (e.g., microbubbles, perfluorocarbon droplets) can be administered to the patient prior to and/or during delivery of the energy to the clot material to facilitate disruption of the obstruction. For example, such a cavitation-facilitating agent can be administered to the patient intra-arterially via a need1e and/or catheter, and carried to the clot material via blood flow. In one form of administration via catheter, the agent may be delivered through a catheter whose distal tip is placed at or near the proximal face of the clot material.

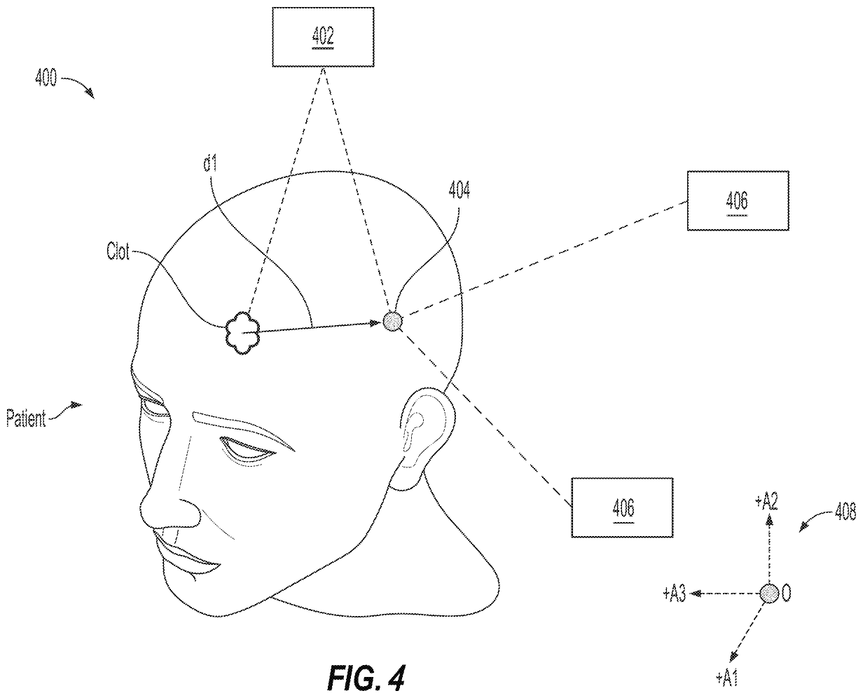

FIG. 4 depicts a detection system 400 in accordance with some embodiments of the present technology. In some embodiments, the system 400 can be similar to any of the embodiments of the systems disclosed herein, except as further described. As shown in FIG. 4, the detection system 400 may comprise a first data collector 402, a reference marker 404, and a second data collector 406. According to some embodiments, the second data collector 406 comprises one or more optical cameras. For example, as shown in FIG. 4, the second data collector 406 may comprise at least two optical cameras. In some embodiments, the second data collector 406 comprises at least two optical cameras for each reference marker 404. In some embodiments, the second data collector 406 comprises an optical motion capture system similar to the optical motion capture systems developed for gait analysis or film animation.

As described herein, the reference marker 404 may comprise properties based, at least in part, on the modality of the second data collector 406. For example, a reference marker 404 configured for use with a second data collector 406 comprising optical cameras may be retroreflective or may actively emit light (e.g., infrared light). The reference marker 404 may be configured to be removably adhered or coupled to the patient's head, as shown in FIG. 4. A location of the reference marker 404 may be selected, at least in part, on anatomy of the patient, the position of the patient within the treatment environment, the position of the second data collector 406, and/or the position of the first data collector 402.

According to some embodiments, for example as shown in FIG. 4, the second data collector 406 is configured to obtain data characterizing a position of the reference marker 404 relative to a coordinate system 408 of the treatment environment. As shown in FIG. 4, the coordinate system 408 may comprise an origin O, a first axis A1, a second axis A2, and a third axis A3 (collectively “axes A”). For example, a treatment environment comprising a rear compartment of an ambulance (e.g., a generally rectangular room) may have a coordinate system 408 comprising an origin O at a lower, front, right corner of the rear compartment, with axis A1 extending away from the origin O toward the back of the rear compartment, axis A2 extending away from the origin O toward the ceiling of the rear compartment, and axis A3 extending away from the origin O toward the left side of the rear compartment. In some embodiments, the second data collector 406 can be calibrated such that the second data collector 406 is configured to obtain data characterizing the position of the reference marker 404 relative to the coordinate system 408. Accordingly, the data characterizing the position of the reference marker 404 can comprise three-dimensional (3D) coordinates of the reference marker 404 within the treatment environment.

As described elsewhere herein, the first data collector 402 may comprise any suitable device or collection of devices configured to obtain data characterizing a position of a clot in a blood vessel of a patient. For example, the first data collector 402 may be a CT scanner. In some embodiments, the detection system 400 may further comprise a marking agent configured to facilitate detecting the clot and obtaining data characterizing the position of the clot material by the first data collector 402.

As shown in FIG. 4, the first data collector 402 may be configured to obtain data characterizing a position of the clot relative to the position of the reference marker 404. The data characterizing the position of the clot relative to the position of the reference marker 204 may comprise a 3D position vector d1, as shown in FIG. 4. The detection system 400 and/or the first data collector 402 may be configured to determine the position of the clot relative to the coordinate system 408 of the treatment environment from the position vector d1 and the 3D coordinates of the reference marker 404 relative to the coordinate system 408. For example, a computing device of the detection system 400 and/or the first data collector 402 may be configured to perform mathematical calculations to compute the position of the clot relative to the coordinate system 408 of the treatment environment from the position vector d1 and the data characterizing the position of the reference marker 404 relative to the coordinate system 408. An energy delivery device of the present technology may be configured to receive the data characterizing the position of the clot relative to the coordinate system 408 of the treatment environment, as described herein.

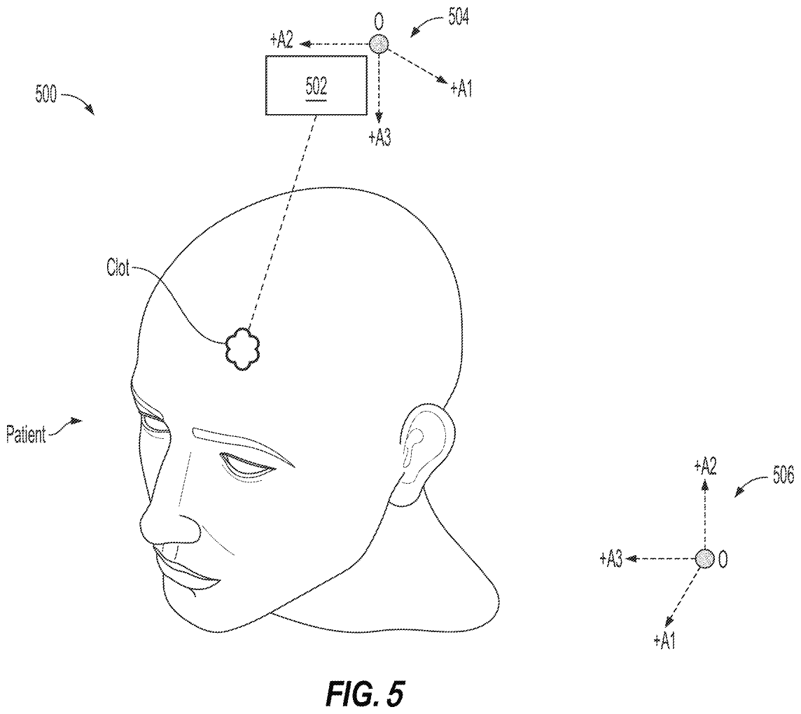

In some embodiments, for example as shown in FIG. 5, a detection system 500 of the present technology comprises a data collector 502 configured to obtain a position of a clot in a blood vessel of a patient without a reference marker and/or a second data collector. (In some embodiments, the system 500 can be similar to any of the embodiments of the system 100 or the system 400 disclosed herein, except as further described.) The data collector 502 may be configured to obtain local data characterizing the position of the clot relative to a local coordinate system 504 of the data collector 502. As shown in FIG. 5, the local coordinate system 504 may be translated and/or rotated relative to a global coordinate system 506 of the treatment environment. Accordingly, to obtain global data characterizing the position of the clot relative to the global coordinate system 506, the data collector 502 can be calibrated to determine a relationship between the local coordinate system 504 and the global coordinate system 506. In some embodiments, the relationship can include a transformation matrix or the like. The calibration may involve comparing the local and global data to determine the relationship between the local and global coordinate systems 504, 506. In some embodiments, calibration of the data collector 502 is performed prior to detecting and/or treating the thrombus. Alternatively or additionally, calibration may be performed during the process of detecting and/or treating the blood clot. The calibration may be performed by a human operator and/or a computing device. The detection system 500 can be configured to obtain the local data characterizing the position of the clot relative to the local coordinate system 504 and, based on the relationship between the local coordinate system 504 and the global coordinate system 506 of the treatment environment, compute the global data characterizing the position of the clot relative to the global coordinate system 506 of the treatment environment, which may in turn be received by an energy delivery device.

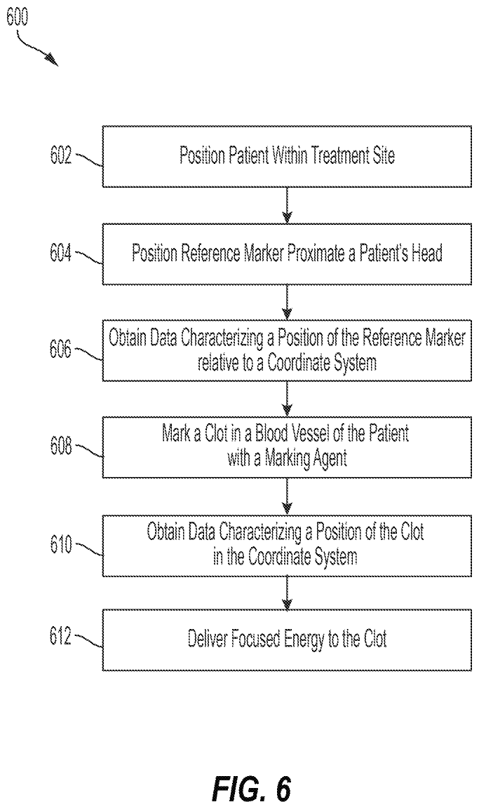

FIG. 6 is a flow diagram of a process 600 for detecting and disrupting a clot in a blood vessel of a patient in accordance with several aspects of the present technology. The process 600 may, but need not, be performed with any of the suitable embodiments of the system 100, 400, 500 disclosed herein, including optionally any suitable embodiments of the energy delivery device 300. The particular processes described herein are exemplary only and may be modified as appropriate to achieve the desired outcome. In various embodiments, other suitable methods or techniques can be utilized for thrombolysis. Moreover, although various aspects of the methods disclosed herein refer to sequences of steps, in various embodiments the steps can be performed in different orders, two or more steps can be combined together, certain steps may be omitted, and additional steps not expressly discussed can be included in the process as desired.

As noted above, a system for detecting and disrupting a clot in a blood vessel of a patient of the present technology may comprise a treatment environment including a detection system and an energy delivery device. In some embodiments, the treatment environment is mobile (e.g., a vehicle) such that the process 600 can be performed at a point of care remote from a hospital or clinic, thus minimizing adverse events associated with delays in treatment and the associated complications and adverse outcomes. As shown in FIG. 6, the process can begin at block 602 with positioning a patient within the treatment environment. In some embodiments, a positioning device (e.g., bed, chair, etc.) is used to maintain the patient at a fixed location and/or orientation within the treatment environment. Additionally or alternatively, a head immobilizer may be utilized to maintain a head of the patient at a fixed position and/or location within the treatment environment. The process 600 can continue at block 604 with positioning one or more reference markers proximate a head of the patient. For example, the reference markers may be placed over the frontal bone, the parietal bone, the temporal bone, or others. In some embodiments, the reference markers are removably coupled or adhered to the patient. Additionally or alternatively, the reference markers may be placed within the environment surrounding the patient.

The process can proceed at block 606 with obtaining reference data (e.g., 3D coordinates) characterizing a position of each reference marker relative to a coordinate system of the treatment environment. In some embodiments, the reference data is obtained using the detection system. As previously described, the detection system can comprise a second data collector configured to obtain the reference data. Alternatively or additionally, the reference data may be obtained by a first data collector of the detection system. In some embodiments, the obtained reference data directly characterizes a position of each reference marker relative to a coordinate system of the treatment environment. In some embodiments, obtaining the reference data comprises obtaining local reference data characterizing a position of each reference marker relative to a local coordinate system of the second data collector. The local reference data may be converted to the reference data (e.g., by applying a transformation matrix obtained via calibration of the second data collector or first data collector).

In some embodiments, the process 600 includes block 608 in which the blood clot is marked with a marking agent. The marking agent may be administered intravenously to the patient upstream of the clot such that the marking agent travels downstream through the patient's vasculature until it reaches the clot and is positioned proximate the clot. As previously described, the marking agent can comprise a nanoparticle, a biomarker, a contrast agent, or another suitable material for facilitating detection of the clot.

The process 600 may proceed at block 610 with obtaining clot data characterizing a position of the clot in the coordinate system of the treatment environment. In some embodiments, the clot data is obtained using the detection system. As described elsewhere herein, the detection system can comprise a first data collector configured to obtain the clot data. Obtaining the clot data may comprise obtaining 3D coordinates of the clot and/or the reference markers to determine a position vector defining the distance between the position of the clot and the positions of the reference markers. The reference data (e.g., the 3D coordinates of each of the reference markers relative to the coordinate system of the treatment environment) and the position vector can be used to determine 3D coordinates of the clot relative to the coordinate system of the treatment environment (e.g., the clot data). In some embodiments, obtaining the clot data comprises obtaining local clot data characterizing the position of the clot relative to a local coordinate system of the first data collector and converting the local clot data into clot data (e.g., data characterizing the position of the clot relative to the coordinate system of the treatment environment) based on a relationship between the coordinate systems determined by calibration.

In some embodiments, focused energy is delivered to the clot to fragment the clot at block 612. The clot data obtained in block 610 can be received by an energy delivery device and a position, orientation, and/or parameter of the energy delivery device can be modified such that a position of a focal point of the focused energy is the same as the position of the clot within the treatment environment. Additionally or alternatively, a position and/or orientation of the patient can be modified to align the focal point with the clot. As described elsewhere herein, in some embodiments the energy delivery device is configured to deliver HIFU energy to the clot. The focused energy can induce fragmentation of the clot in order to clear the obstructing clot and restore blood flow to the affected blood vessel. In some embodiments, a thrombolytic agent (e.g., tPA, itPA) can be administered to the patient before, during, and/or after the delivery of energy to the clot. As previously described, a cavitation-facilitating agent may be administered to the patient before or during the delivery of energy to the clot to facilitate disruption of the clot.

### Conclusion

Although many of the embodiments are described above with respect to systems, devices, and methods for detecting and disrupting obstructions such as clot material in cerebral blood vessels, the technology is applicable to other applications and/or other approaches, such as peripheral thrombolysis, thermal ablation, or targeted drug delivery. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-6.

As used herein, the terms “generally,” “substantially,” “about,” and similar terms are used as terms of approximation and not as terms of degree, and are intended to account for the inherent variations in measured or calculated values that would be recognized by those of ordinary skill in the art.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method comprising:
positioning a reference marker proximate a head of a patient, the patient having a clot within a cerebral blood vessel;
obtaining first data characterizing a position of the reference marker relative to a coordinate system;
obtaining second data characterizing a position of the clot relative to the position of the reference marker;
based on the first data and the second data, determining a position of the clot relative to the coordinate system; and
based on the position of the clot relative to the coordinate system, delivering focused energy from an extracorporeally-positioned energy delivery device to the clot, thereby fragmenting the clot.

2. The method of claim 1, wherein the first data comprises three-dimensional coordinates and the second data comprises a three-dimensional position vector.

3. The method of claim 1, wherein the reference marker is a first reference marker, the method further comprising positioning second and third reference markers proximate the head of the patient.

4. The method of claim 1, wherein obtaining the first data comprises obtaining an image of the reference marker using an optical camera system comprising at least two cameras.

5. The method of claim 4, wherein the reference marker is retroreflective.

6. The method of claim 1, wherein obtaining the first data comprises determining three-dimensional coordinates of the reference marker using a position sensing device.

7. The method of claim 1, wherein obtaining the first data comprises obtaining an image of the reference marker using a medical imaging device.

8. The method of claim 7, wherein the medical imaging device includes a modality comprising x-ray, fluoroscopy, magnetic resonance imaging, computed tomography, ultrasound, positron emission tomography, single photon emission coherence tomography, optical coherence tomography, magnetic particle imaging, or magnetic particle spectroscopy.

9. The method of claim 8, wherein obtaining the second data comprises obtaining an image of the patient using the medical imaging device.

10. The method of claim 1, further comprising marking the clot with a marking agent.

11. The method of claim 10, wherein marking the clot comprises intravenously administering the marking agent to the patient.

12. The method of claim 10, wherein the marking agent comprises a biomarker, a nanoparticle, or a contrast agent.

13. The method of claim 1, wherein positioning the reference marker, obtaining the first data, obtaining the second data, determining the position of the clot relative to the coordinate system, and delivering the focused energy occurs in a vehicle.

14. The method of claim 1, further comprising administering a fibrinolytic agent to the patient before, during, or after delivery of the focused energy.

15. The method of claim 1, further comprising administering a cavitation-facilitating agent to the patient prior to or during delivery of the focused energy.

16. The method of claim 1, wherein the energy delivery device is a high-intensity focused ultrasound device.

17. The method of claim 1, further comprising modifying a position or an orientation of the patient based on the position of the clot relative to the coordinate system.

18. The method of claim 1, further comprising modifying a position or an orientation of the energy delivery device based on the position of the clot relative to the coordinate system.

19. The method of claim 1, further comprising modifying a parameter of the energy delivery device based on the position of the clot relative to the coordinate system.

20. The method of claim 19, wherein the parameter comprises a frequency, an acoustic power, a pulse width, a pulse duration, a number of pulses, or a treatment duration.

21. A method comprising:
positioning a patient within a treatment environment, the patient having a clot within a blood vessel;
marking the clot with a marking agent;
determining a relationship between a local coordinate system of a detection system and a global coordinate system of the treatment environment;
obtaining data characterizing a position of the clot relative to the global coordinate system of the treatment environment with the detection system;
based on the data, delivering focused energy from an extracorporeally-positioned energy delivery device to the clot, thereby disrupting the clot.

22. The method of claim 21, wherein obtaining the data characterizing the position of the clot relative to the global coordinate system of the treatment environment comprises obtaining local data characterizing the position of the clot relative to the local coordinate system of the detection system and, based on the relationship between the local and global coordinate systems, determining the position of the clot relative to the global coordinate system.

23. The method of claim 21, wherein the relationship comprises a transformation matrix.

24. The method of claim 21, wherein the data comprises three-dimensional coordinates.

25. The method of claim 21, wherein the detection system comprises a medical imaging device.

26. The method of claim 21, wherein the marking agent comprises a peptide, a nanoparticle, or a contrast agent.

27. The method of claim 21, wherein the energy delivery device is a high-intensity focused ultrasound device.

28. A non-transitory computer readable medium having stored thereon instructions executable by a computing device to cause the computing device to perform functions comprising:
obtaining first data characterizing a position of a reference marker relative to a coordinate system;
obtaining second data characterizing a position of a blood clot within a blood vessel of a patient relative to the position of the reference marker;
based on the first data and the second data, determining a position of the blood clot relative to the coordinate system; and
based on the position of the blood clot relative to the coordinate system, causing an extracorporeally-positioned energy delivery device to deliver focused energy to the blood clot to fragment the clot.

29. The non-transitory computer-readable medium of claim 28, wherein the first data comprises three-dimensional coordinates and the second data comprises a three-dimensional position vector.

30. The non-transitory computer-readable medium of claim 28, wherein the first data and the second data each comprise three-dimensional coordinates.

31. The non-transitory computer-readable medium of claim 28, wherein causing the energy delivery device to deliver focused energy to the blood clot comprises modifying a position of the energy delivery device.

32. The non-transitory computer-readable medium of claim 28, wherein causing the energy delivery device to deliver focused energy to the blood clot comprises modifying a parameter of the energy delivery device.
